(19) United States
(12) Patent Application Publication
(10) Pub. No.: US 2009/0263416 A1
(43) Pub. Date: Oct. 22, 2009

(54) REDUCTION OF ANTIBIOTIC RESISTANCE IN BACTERIA

(76) Inventors: Karl A. Dawson, Lexington, KY (US); Melissa C. Newman, Corinth, KY (US)

Correspondence Address: KING & SCHICKLI, PLLC, 247 NORTH BROADWAY, LEXINGTON, KY, 40507, US

(21) Appl. No.: 12/305,156

(22) PCT Filed: Jun. 18, 2007
(86) PCT No.: PCT/US07/14281

§ 371 Date: Apr. 8, 2009

Related U.S. Application Data
(60) Provisional application No. 60/814,236, filed on Jun. 16, 2006.

(51) Int. Cl. A61K 36/06; C12N 1/20

(52) U.S. Cl. 424/195.16; 435/252.1; 435/252.8; 435/252.7

(57) ABSTRACT

In one aspect of the present invention, a method is provided for reducing or eliminating antibiotic resistance in bacteria, comprising exposing the bacteria to a composition comprising a yeast cell wall preparation in an amount effective for reducing or eliminating resistance of the bacteria to at least one antibiotic. In one embodiment, the method comprises exposing the bacteria to the yeast cell wall preparation in an amount effective for eliminating a plasmid conferring resistance to the antibiotic, or for preventing transfer of the plasmid between bacteria. In another aspect, a method is provided for reducing prevalence of antibiotic-resistant bacteria in an animal, comprising administering to the animal a composition comprising a yeast cell wall preparation in an amount effective for reducing or eliminating the presence of an antibiotic-resistant bacterial population in the animal.

This application claims the benefit of priority in U.S. provisional patent application Ser. No. 60/814,236 filed on Jun. 16, 2006, the entirety of the disclosure of which is incorporated herein by reference.

### TECHNICAL FIELD

The present invention relates to methods for reducing the prevalence of antibiotic resistance in bacteria. In particular, the invention relates to use of a yeast cell wall preparation (YCWP) to reduce antibiotic resistance, and to restore sensitivity of bacterial organisms to antibiotics.

### BACKGROUND OF THE INVENTION

Bacterial antibiotic resistance is a significant issue faced by various industries, including the food and agricultural industries, the medical and veterinary professions, and others. The potential for transfer of antibiotic resistance, or of potentially lethal antibiotic-resistant bacteria, for example from a food animal to a human consumer, is of particular concern.

Current methods for controlling development and spread of antibiotic-resistant bacteria include changes in antibiotic usage and patterns of usage of different antibiotics, increased governmental surveillance and regulation, and continued development of new and improved antibiotics. However, the ability of most bacteria to adapt to antibiotic usage and to acquire resistance to existing and new antibiotics often overcomes such conventional measures, and requires the continued development of alternative means for control of antibiotic resistance in bacteria.

Excessive use of, for example, antibiotic growth promoters in animal feeds imposes a selection pressure for bacteria that are resistant to such antibiotics. As a result of such concerns, certain government organizations have imposed a ban on such antibiotic-growth promoters. Animal producers have had to modify their practices to reduce animal stress and therapeutic prescription of antibiotics, as well as search for alternatives providing comparable health and economic benefits. Thus, a need exists for alternative methods for reducing harmful effects of certain bacterial organisms on livestock, while at the same time reducing the risk of dissemination of antibiotic resistance among pathogenic and commensal bacteria.

Alternative means for overcoming the tendency of bacteria to acquire resistance to antibiotic control measures have taken various forms. For example, it is known to use various food preservation methods (pH, a_{w}, temperature, oxidation-reduction potential, and the like) to create a series of “hurdles” to prevent microbial growth and reproduction, and to reduce the threat of spread of bacterial resistance in a food-processing environment. It is also known to control bacterial adhesion to thereby control biofilm formation and development of resistance. Similarly, in the human food and animal feed industries, modulation of gut microflora has been evaluated for beneficial effects on reducing pathogen load without resort to antibiotics. Currently, probiotics, prebiotics, and combinations thereof are used by the food industry as components of functional foods intended to reduce pathogen load and improve the health of the digestive system, potentially via a competitive exclusion effect.

Such alternative means are generally effective for their intended purpose However, the continued concern in the food and food animal industries regarding bacterial acquisition of antibiotic resistance, and the potential for transfer of antibiotic-resistant bacteria from food sources to humans consuming them, points to the continued need for development of alternative means for control of antibiotic resistance.

One method evaluated for control, that is, reducing or removing antibiotic resistance is so-called “curing” of antibiotic resistance. Antibiotic resistance information in the bacterial cell is most often located on plasmids and extra-chromosomal elements (Lakshmi, 1987). Thus, elimination of such drug-resistance plasmids results in loss of antibiotic resistance by the bacterial cell. “Curing” of a microorganism refers to the ability of the organism to spontaneously lose a resistance plasmid under the effect of particular compounds and/or environmental conditions, thus reverting to the antibiotic-sensitive state (Trevor, 1986).

As examples, sodium dodecyl sulfate (SDS), antibiotics, thymine starvation, quinine, elevated temperature, and combinations have been evaluated as “curing systems” for sensitization of antibiotic-resistant bacteria (Chakrabartty et al., 1984; Hahn and Ciak, 1976; Gupta et al., 1980; Obaseiki-Ebor, 1984; Poppe and Gyles, 1988; Reddy et al., 1986). The present inventors have surprisingly found that preparations comprising yeast cell wall (hereinafter yeast cell wall preparations or YCWP) reduce the prevalence of antibiotic resistance in bacteria. Without wishing to be bound by any theory, it is hypothesized that such YCWP may have a curative or “curing” effect on previously antibiotic resistant bacteria, potentially by reduction in antibiotic-resistance plasmids and/or prevention of plasmid transfer.

### SUMMARY OF THE INVENTION

The present invention addresses the above-identified need in the art by providing a method for reducing or eliminating antibiotic resistance in bacteria, comprising exposing the bacteria to a composition comprising a yeast cell wall preparation in an amount effective for reducing or eliminating resistance of the bacteria to at least one antibiotic. The yeast cell wall preparation may be included in the composition in an amount effective for reducing or eliminating the presence of a bacterial plasmid which confers resistance to that antibiotic. Still further, the yeast cell wall preparation may be included in the composition in an amount effective for preventing or reducing the transfer between bacteria of a plasmid which confers resistance to said antibiotic.

In one embodiment of the present invention, the yeast cell wall preparation may be included in the composition in an amount of from about 0.01% (w/v) to about 1.0% (w/v). Typically, the yeast cell wall preparation is derived from a species selected from the group consisting of Saccharomyces, Candida, Kluyveromyres, Torulaspora, and mixtures thereof. The antibiotic may be one or more of ampicillin, bacitracin, clindamycin, gentamycin, erythromycin, kanamycin, penicillin, streptomycin, tetracycline, trimethoprim, chloramphenicol, sulfamethazole, and vancomycin. The bacteria may be selected from various groups of bacteria, including normal enteric bacteria, enteric pathogens, disease-causing organisms, and combinations or mixtures thereof.

In another aspect of the present invention, a method for reducing prevalence of antibiotic resistant bacteria in an animal is provided, comprising administering to the animal a composition comprising a yeast cell wall preparation in an amount effective for reducing or eliminating the presence of an antibiotic-resistant bacterial population in said animal. The yeast, bacteria, and antibiotics may be as set forth above. The bacteria may be any bacteria which is or is capable of becoming a pathogen of the animal intestinal tract. Embodiments of the invention include compositions for administration as dietary supplements, and compositions formulated for admixing with a feed ration. In one embodiment, the composition for reducing prevalence of antibiotic-resistant bacteria is administered to the animal in an amount providing yeast cell wall preparation at from about 0.1 to about 10 kg/T of feed.

The composition may be formulated for feeding to bovine, porcine, avian, equine, ovine, lapine, and caprine species, using methods and ingredients known to the skilled artisan. The avian species may be selected from various production birds including chicken, turkey, duck, goose, pheasant, and quail, or may be a companion bird.

As should be appreciated, the embodiments shown and described herein are an illustration of one of the modes best suited to carry out the invention. It will be realized that the invention is capable of other different embodiments and its several details are capable of modification in various, obvious aspects all without departing from the invention. Accordingly, the drawings and descriptions will be regarded as illustrative in nature, and not as restrictive. Unless otherwise indicated, all patents, patent applications, and non-patent documents referenced in the present disclosure are incorporated herein by reference in their entirety.

### BRIEF DESCRIPTION OF THE DRAWINGS

The accompanying drawings incorporated in and forming a part of the specification, illustrate several aspects of the present invention and together with the description serve to explain the principles of the invention. In the drawings:

FIG. 1 shows the effect of purified yeast cell wall preparation (P-YCWP) on the growth of plasmid-containing E. coli;

FIG. 2 shows the percent curing of streptomycin-resistant S. enteritidis (ATCC 13076) following treatment with yeast cell wall preparation (YCWP), P-YCWP, ethidium bromide (EB), and iododeoxyuridine (IDU);

FIG. 3 shows percent curing of streptomycin and ampicillin-resistant Salmonella montevideo following treatment with yeast cell wall preparation (YCWP), P-YCWP, EB, and IDU;

FIG. 4 shows recovery of antibiotic sensitivity in E. coli XL1-Blue following treatment with P-YCWP, EB, and IDU;

FIG. 5 shows growth of ampicillin-resistant Salmonella spp. on ampicillin-containing medium following exposure to P-YCWP;

FIG. 6 shows growth of streptomycin-resistant Salmonella spp. on ampicillin-containing medium following exposure to P-YCWP;

FIG. 7 shows growth of ampicillin-resistant Salmonella spp. on ampicillin- or streptomycin-containing medium following exposure to P-YCWP;

FIG. 8 shows growth of ampicillin-resistant Salmonella spp. on ampicillin- or streptomycin-containing medium following exposure to P-YCWP;

FIG. 9 shows effect of mannose on antibiotic sensitivity of Salmonella spp. by replica plating technique;

FIG. 10 shows transconjugant formation in vitro during E. coli XL1-Blue (donor) and during E. coli MC1000 (recipient) mating in the presence of P-YCWP;

FIG. 11 shows transconjugant formation in vitro during E. coli XL1-Blue (donor) and during E. coli MC1000 (recipient) mating in the presence and absence of YCWP;

FIG. 12 shows transconjugant formation in vitro during E. coli XL1-Blue (donor) and during E. coli MC1000 (recipient) mating with addition of yeast cell wall preparation at 60 and 120 minutes of incubation;

FIG. 13 shows transconjugant formation in swine fecal samples in the presence and absence of P-YCWP;

FIG. 14 shows transconjugant formation in swine fecal samples in the presence of different mannan-containing compounds (0.3%);

FIG. 15 shows transconjugant formation in swine fecal samples in the presence of different mannan-containing compounds (0.5%);

FIG. 16 shows reduction in the tet A resistance gene in cecal samples of chickens provided a yeast cell wall preparation containing composition (BIO-MOS, Alltech, Inc.);

FIG. 17 shows reduction in the tet A resistance gene in cecal samples of turkeys provided a yeast cell wall preparation containing composition (BIO-MOS, Alltech, Inc.);

FIG. 18 shows reduction in the tet B resistance gene in cecal samples of turkeys provided a yeast cell wall preparation containing composition (BIO-MOS, Alltech, Inc.); and

FIG. 19 shows reduction in the tet M resistance gene in cecal samples of turkeys provided a yeast cell wall preparation containing composition (BIO-MOS, Alltech, Inc.).

Reference will now be made in detail to the present preferred embodiment of the invention, an example of which is illustrated in the accompanying drawings.

### DETAILED DESCRIPTION OF THE INVENTION

The present invention addresses the above-identified need in the art by providing a method for reducing the prevalence of or eliminating antibiotic resistance in bacterial populations, comprising exposing the bacteria to a composition comprising an effective amount of a yeast cell wall preparation. In one embodiment, the yeast cell wall preparation is contained in the composition in an amount effective for reducing or eliminating a plasmid conferring resistance to an antibiotic to the bacteria. In another aspect, the present invention provides a method for increasing susceptibility to an antibiotic in bacteria, comprising exposing the bacteria to a composition comprising a yeast cell wall. Unless otherwise indicated, all cited patents, published patent applications, and non-patent documents are incorporated into the present disclosure in their entirety by reference.

### GENERAL EXPERIMENTAL METHODS

### Isolates and materials

Unless otherwise indicated, the experimental materials and methods recited herein apply to the entirety of the present disclosure, and to all aspects and embodiments of the present invention. Gram-negative bacterial isolates were obtained from the University of Kentucky. Plasmid-containing Salmonella species were obtained from the culture collection maintained by Alltech, Inc. (Nicholasville, Ky.). Isolates and their antibiotic susceptibility patterns are presented in Tables 1 and 2.

TABLE 1
Antibiotic resistance and plasmid profiles of gram-negative isolates.

| Strains | Plasmid^{a} | Plasmid Size (Kb)^{a} | Antibiotic Resistance^{a} |
|---|---|---|---|
| E. coli (CS2) | 4 | 7, 10, 13, >13 | AM, VA, B, G, CC, N, TMP, |
| | | | TE, P, S, K |
| E. coli (CS3) | 3 | 6, 8.4, 12 | AM, VA, B,, CC, N(I), TMP, |
| | | | E, TE, P, S, K |
| E. coli (CS6) | 6 | 1, 2.7, 3.8, 4.5, 5, 13 | VA, B, G, CC, P(I), S(I) |
| E. coli (CS9) | 2 | 13, >13 | VA, B, G, CC, P, S(I) |
| E. coli (CS7) | 6 | 1, 2.7, 3.6, 4.5, 5, 13 | VA, B, G, CC, E(I), TE, P, S(I) |
| E. coli (CS15) | 6 | 1, 2.7, 3.6, 4.5, 5, 14 | VA, B, G, CC, P, S(I) |
| E. coli (CS16) | 6 | 1, 2.7, 3.6, 4.5, 5, 15 | VA, B, G, CC, P(I), S(I) |
| E. coli (CS20) | 5 | 1.3, 3, 3.5, 8, 13 | AM, VA, B, G, CC, E(I), |
| | | | TE, 0, S(I) |
| Providencia rettgeri (CS21) | 0 | | VA, B, G, CC, E, TE, |
| | | | P(I), CF, K(I) |
| Proteus vulgaris (CS35) | 0 | | VA, B, CC, E, TE, CF |
| Proteus vulgaris (CS36) | 0 | | AM, VA, B, G, CC, E(I), |
| | | | TE, P, S, K |
| E. coli O157:H7 | 2 | 2.2, 13 | VA, B, CC, E(I), P(I), |
| E. coli (P 6-2) | 4 | 2.4, 3.2, 4.5, 13 | VA, B, CC, E(I), P |
| E. coli (P 6-3) | 1 | 13 | VA, B, G, CC, |
| | | | N(I), E(I), S, K |
| E. coli (P 6-13) | 1 | 2.5 | AM, VA, B, CC, E, TE, P, S(I) |
| Citrobacter ssp. (P 6-17) | 1 | 1.7 | VA, B, CC, E, TE, P, S |
| Klebsiella pneumonia (P 6-19A) | 2 | 2.3, 13 | AM, VA, B, G, CC, E(I), |
| | | | TE, P, S(I) |
| Klebsiella pneumonia (P 6-19C) | 2 | 2.3, 13 | AM, VA, B, CC, N(I), P, K |
| E. coli (P 7-3) | 3 | 2.5, 2.7, 3.6 | AM, VA, B, G, CC, E(I), |
| | | | TE, P, S |
| E. coli (P 7-4) | 2 | 1.8, 3.5 | VA, B, CC, E(I), P(I) |
| E. coli (P 7-5) | 5 | 2.5, 2.7, 3.6, 5.8, 13 | AM, VA, B, G, CC, |
| | | | E(I), TE, P, S(I) |
| E. coli (P 7-9) | 5 | 2.5, 2.7, 3.6, 5.8, 14 | AM, VA, B, G, CC, E(I), |
| | | | TE, P, S(I) |
| E. coli (P 8-4) | 0 | | AM, VA, B, CC, E(I), TE, |
| | | | P |
| E. coli (P 8-6) | 2 | 1.8, 3.5 | AM, VA, B, CC, E(I), TE, |
| | | | P, S(I) |
| E. coli (P 9-3) | 1 | 1.7 | VA, B, CC, AMC(I), E(I) P |
| E. coli (P 9-9) | 0 | | AM, VA, B, CC, E(I), TE, |
| | | | P, S(I) |
| E. coli (P 10-4) | 4 | 1.8, 2.5, 3.5, 5 | VA, B, CC, E(I), P(I) |
| E. coli (P 10-6) | 2 | 3.5, 1.8 | VA, B, CC, E(I), P(I) |

^{aPlasmid number and size determined by Plamid Miniprep System and gel electrophoresis.}
Antibiotic resistance determined by Kirby Bauer sensitivity test.

TABLE 2
Antibiotic resistance and plasmid profiles of Salmonella spp.

| | | Plasmid Size | |
|---|---|---|---|
| Strains | # of Plasmids | (Kb) | Antibiotic Resistance |
| S. agona | 2 | 2, 13 | L, VA, CC, AM, S(I) |
| S. boedeney | 2 | 13, .13 | L, VA, CC, AM |
| S. brand | 1 | 13 | L, NA, VA, CC, AM |
| S. choleraesuis, subsp. | 2 | 13, >13 | L, VA, CC, AM |
| arizonae (ATCC 13314) | | | |
| S. choleraesuis subsp. | 1 | 13 | L, VA, CC, AM |
| cholerasuis (ATCC 13312) | | | |
| S. dublin | 1 | 13 | L, VA, CC, AM |
| S. enteritidis (ATCC 13076) | 3 | 2, 2.5, 13 | L, G, VA, CC, S |
| S. cholerasuis | 1 | 3 | L, VA, CC, AM |
| S. heldelberg | 3 | 2, 13, >13 | L, VA, CC, AM, S(I) |
| S. kedongon | 1 | 13 | L, NA, VA, CC, AM(I) |
| S. kentucky | 1 | 13 | L, VA, CC, TE, S |
| S. monterido | 7 | 1, 1.5, 2, 2.5, | L, VA, CC, AM, K, GM, S(I) |
| | | 3.5, 7, 13 | |
| S. pullorum (ATCC 19945) | 2 | 1.3, >13 | L, VA, CC |
| S. schwarzengrud | 1 | 1 | L, VA, CC, AM, S(I) |
| S. senft | 4 | 2, 2.5, 7, 13 | L, VA, CC, K, GM, S(I) |
| S. typhi | 1 | 13 | L, VA, CC, AM, S(I) |

Escherichia coli XL1-blue (Stratagene, La Jolla, Calif.), E. coli RK2 (ATCC 33766) containing broad host range plasmids (pRK248clts) possessing a tetracycline resistant determinant (TetR), and E. coli MC1000 (ATCC 37221) possessing an ampicillin resistant marker (ampR) were obtained from the American Type Culture Collection (Manassas, Va.).

### Yeast Cell Wall Preparations

The yeast cell wall preparations evaluated herein are set forth in Table 3, and were obtained from Alltech, Inc.

TABLE 3
Composition of yeast cell wall preparations.

| | | Manan (%) | | |
|---|---|---|---|---|
| Sample | | (as | Glucan (%) | |
| Identification | *Protein (%) | Mannose) | (as Glucose) | Observations |
| Purified- | 34 | 14 | 38 | YCWP from S. cerevisiae. (water soluble) |
| YCWP^{a}(PYCWP) | | | | |
| Non-purified- | 12.5 | 12 | 34.8 | YCWP from S. cerevisiae. (water insoluble) |
| YCWP (YCWP) | | | | |
| A | 3.125 | 85 | 0 | Alfa-mannan from S. cerevisiae |
| B | 0 | 0 | 98 | Beta 1,6-Glucan from S. cerevisiae |
| C | 4.4375 | 60 | 0 | Beta-mannan from plant material |
| D | 0 | 39.3 | 39.6 | Mannan from YCW^{b} from S. cerevisiae |
| E | 39.4375 | 21.4 | 41 | Mannan from YCW from S. cerevisiae |
| F | 35.625 | 18.6 | 40.3 | Beta 1,6-Glucan |
| G | 11.125 | 14.7 | 80.4 | Beta 1,6-Glucan |
| H | 34.0625 | 37 | 10 | Mannan from YCW from S. cerevisiae |
| I | 33.8125 | 37.2 | 10.4 | Soluble mannan fraction |
| J | 33.5 | 48.6 | 14.1 | |
| K | 12.875 | 12.1 | 51.9 | |
| L | 39 | 32 | 22 | Mannan 50:50 soluble and insoluble |

*Nitrogen data provided by Alltech Inc. Protein = N*6.25)
^{a}YCWP Yeast Cell Wall Preparation
^{b}YCW Yeast cell wall

Antibiotics for sensitivity testing were obtained from BBL Laboratories (Cockeysville, Md.), and included ampicillin, bacitracin, clindamycin, gentamycin, erythromycin, kanamycin, penicillin, streptomycin, tetracycline, trimethoprim, chloramphenicol, sulfamethazole, and vancomycin. Purified carbohydrates were obtained from Difco (Detroit, Mich.).

### Bacterial Growth Rate

Bacterial growth rate was evaluated by inoculating the desired isolate into Mueller Hinton Broth (MHB) containing one of the following; 0.1% glucose, purified yeast cell wall preparation (P-YCWP) at 0, 0.01, or 0.1%, or non-purified YCWP at 0, 0.01, or 0.1%. Growth was measured by a turbidimetric method at 0, 6, and 12 hour intervals. Optical density of each sample was monitored using a BioMate 3 spectrophotometer at 600 nm ((National Committee for Clinical Laboratory Standards (NCCLS) 2000; Morehead and Dawson, 1992)).

### Curing

Plasmid curing of antibiotic resistant bacteria was measured as described by Lakshmi and Thomas (1989) and NCCLS (2000). Briefly, aliquots (final inoculum concentration approximately 1×10⁵ CFU/ml) of Salmonella isolates, E. coli isolates, and commercial isolates of E. coli XL1-blue and E. coli RK2 were added to tubes containing 1 ml aliquots of increasing concentrations of P-YCWP or YCWP (0, 0.01, 0.1, 0.3, 0.5, 1.0, 2.0, 3.0% w/v). A control culture was established by adding bacterial isolates as described above to aliquots of increasing concentrations of mannose. Known curing agents [ethidium bromide and iododeoxyuridine (IDU)] were used as positive controls, and to provide a percent curing activity. Tubes were incubated at 35° C. for 24 hr.

Following incubation, bacterial aliquots were plated on MacConkey agar plates (master plates). Antibiotic resistance after treatment was evaluated by replica plating on MacConkey agar containing predetermined concentrations of antimicrobial agents. Minimum curing concentration (MCC) was defined as the minimum concentration of compound able to “cure” bacteria, that is, remove antibiotic resistance, within a 24 hr period. A sample was considered to contain bacteria resistant to an antibiotic when greater than 1% of a sample plated on a master plate of MacConkey agar grew on an antibiotic-containing replica plate.

Antibiotic susceptibility was evaluated by a disk diffusion test (NCCLS, 2000). Resistant colonies from the replica plating experiments were inoculated into MHB, grown to a desired density, and plated on MHB. Antibiotic-impregnated disks were distributed on the MHB plates and the plates were incubated (35° C. for 24 hr). The zone diameters around each disk after incubation were measured, and the organisms were categorized as resistant, intermediate, or susceptible based on NCCLS guidelines.

### Plasmid Evaluation

Plasmids were evaluated by microbial lysis and extraction of DNA (Mini-prep System, Bio-Rad Laboratories, Hercules, Calif.), followed by electrophoretic separation on 1% agarose gel (Bio-Rad). Gels were stained with ethidium bromide (EB) against a DNA molecular weight standard (250 bp to 12 kb ladder, Stratagene). The gels were photographed using a VersaDoc-Imaging System (Bio-Rad) under short wave UV light, filter #1 with 30 sec. exposure.

### Antibiotic Adsorption

Ability of P-YCWP to adsorb antibiotic was evaluated by growing Salmonella spp. in MHB containing antibiotic and increasing concentrations (0, 0.3, 0.5%) of P-YCWP. Plasmid-containing Salmonella spp. were grown in MHB containing filter sterilized antibiotic (ampicillin at 32 μg/ml and streptomycin at 1000 μg/ml) and P-YCWP (0, 0.3, 0.5%). Growth rate was determined using a turbidimetric method as described previously. Spectrophotometric blanking was accomplished using uninoculated medium containing 0.3 or 0.5% P-YCWP.

### Curing Over Time

Percent cure rate (reduction in resistance to antibiotic) over time was evaluated by growing duplicate aliquots of Salmonella spp. in MHB containing 0, 0.3, or 0.5% P-YCWP, with sampling at 0, 2, 4, 6, and 8 hr. Cure rate was determined as previously discussed.

### Agglutination

E. coli and Salmonella isolates were evaluated for ability to agglutinate P-YCWP and YCWP by growing isolates into TIF slants (10 g peptone, 5 g NaCl, 5 g yeast extract and 15 g bacto agar per liter) for 24 hr. Sugar solutions (100 mM) of glucose, mannose, or fructose were prepared in 100 mM phosphate-buffered solution (PBS) at pH 7.2. Yeast cell wall preparations were suspended (1 g YCWP) into 1 liter of PBS. The grown isolates were resuspended in PBS and mixed with 0.1 ml PBS as control or 0.1 ml sugar solution (glucose, mannose, or fructose) to occupy bacterial attachment sites. For adherence tests, 10 μl of P-YCWP or YCWP suspension was placed on a series of microscope slides. The negative control was μl of PBS mixed with YCWP suspension. Bacterial suspension (10 μl) as described above was added, mixed using an orbital shaker, and observed under light microscope (100-1000× magnification). The ability of any of the sugars to inhibit or weaken agglutination was considered evidence that that sugar played a role in agglutination/attachment for that bacteria.

### Conjugation

Conjugation studies in broth were performed substantially as described by Andrup (Andrup et al., 1998; Andrup and Anderson, 1999). Briefly, overnight cultures of donor (XL-Blue E. coli) and recipient (E. coli MC1000) strains were grown in Luria Bertani (LB) medium containing the appropriate antibiotic (tetracycline at 10 μg/ml and ampicillin at 50 μg/ml, respectively). Donor and recipient isolates were diluted into LB medium without antibiotics. The E. coli mating was performed in LB broth without antibiotic, or LB containing 0.3% or 0.5% P-YCWP or YCWP by combining donor cells (0.5 μl per OD₆₀₀ unit) and varying ratios of recipient cells (1:1, 1:1.5, 1:2, 1:2.5, 1:3 donor:recipient ratio) and incubating with shaking (35° C.). Aliquots were removed at intervals (0, 10, 20, 30, 40, 50, 60, 90, 120, 240, 360, 720 min), vortexed, diluted in LB broth, and plated on MacConkey agar containing ampicillin (50 μg/ml) for recipients, MacConkey agar plus tetracycline (10 μg/ml) for donors, and MacConkey agar plus ampicillin plus tetracycline for transconjugants. The number of donor and recipient cells (N₀) were determined at the beginning (t₀) of the mating process. The number of cells (Nᵢ) at any given time (tᵢ) was calculated in accordance with the formula Nᵢ=N₀e^{kti}, wherein k=ln 2/generation time, assuming exponential growth.

Conjugation with YCWP Supplementation

This mating process was conducted as described above, with supplementation of fresh YCWP (0.5%) every hour over a 3 hour period.

### Conjugation in Solid Media

Overnight cultures of donor (E. coli ATCC 33766) containing a broad host range plasmid (pRK248clts) and recipient (E. coli MC1000) strains were grown in LB medium containing the proper antibiotic as described above. Aliquots (100 μl) of the donor and recipient were placed on LB plates without antibiotic, mixed, and incubated for 12 hours. The mixture was then removed from the plate and resuspended into LB broth Aliquots of diluted and undiluted mixture in LB were plated onto MacConkey agar containing tetracycline, ampicillin, or both as described above, and presence of transconjugants was determined as described.

### Conjugative Transfer Rate

Rate of conjugative transfer was measured as the number of transconjugants per donor per minute over a 10 min period in mating broth (Andrup et al., 1998; Andrup and Anderson, 1999).

### Conjugation in Fecal Samples

Fresh fecal samples were obtained from healthy Yorkshire and Landrace crossbred pigs (4 months old) from the University of Kentucky. The pigs were fed a corn:soybean diet not containing antibiotics. Samples were collected in sterile WHIRLPAK bags and placed on ice for transport. Samples were processed according to Kruse and Sorum (1994). Briefly, one g of sample was diluted with 9 ml of LB broth, and an aliquot (100 μl) was plated on: 1) LB agar to ascertain natural flora; 2) LB plus ampicillin (50 μg/ml) for ampicillin-resistant flora; 3) LB plus tetracycline (10 μg/ml) for tetracycline resistant-flora; and 4) LB plus ampicillin plus tetracycline for flora resistant to both antibiotics. Yeast cell wall treatments (0.3% and 0.5% P-YCWP or YCWP) were prepared in LB as follows, LB broth was used as a negative control.

For mating experiments, 100 g of fecal sample was weighed into a sterile stomacher bag (Fisher Scientific, Pittsburgh Pa.). Donor and recipient E. coli were resuspended in LB broth and added to the fecal samples, followed by addition of yeast cell wall treatment or control. Samples were stomached (Seward Stomacher 400, England) for 60 sec and incubated. At intervals (10, 30, 60, 120, 180, 720 min), 10 g of feces were diluted in 90 ml PBS in a sterile stomacher bag, stomached, and aliquots (100 μl) plated on LB agar plus tetracycline plus ampicillin to determine the number of transconjugants. The donor bacteria (E. coli XL-Blue) contained the lac^{q}ZΔM15 gene on the F′ episome, providing blue-white colonies for the screening of conjugated plasmids.

### Example 1

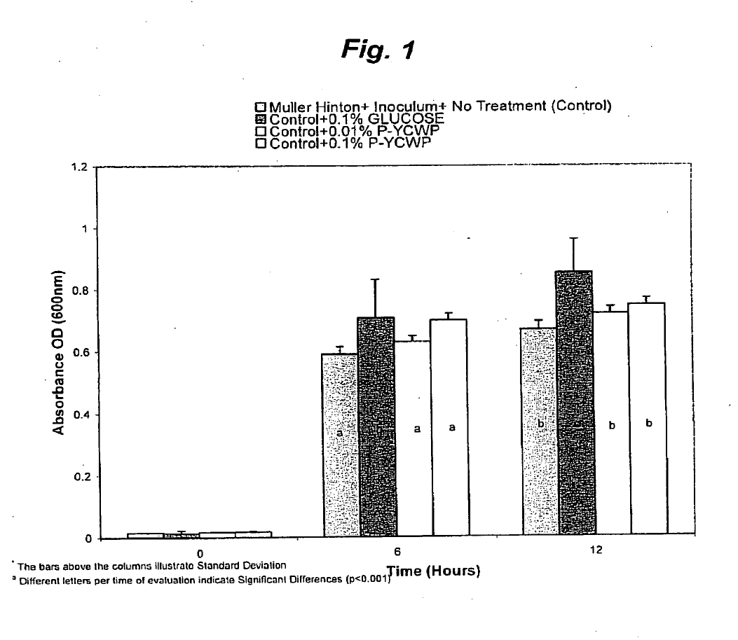

Neither P-YCWP nor YCWP had any effect on growth of E. coli or Salmonella spp. Representative results are provided in FIG. 1. A small but significant effect of YCWP (0.1%) was seen at 6 hr of incubation of the Salmonella spp, but had disappeared by 12 hr (data not shown).

Based on preliminary Kirby Bauer sensitivity test evaluation, the E. coli of swine origin used in the present evaluation demonstrated general multi-drug resistant patterns including 12.8% resistant to 4-5 antibiotics, 15% resistant to 6 antibiotics, and 71% resistant to 7 or more antibiotics. The Salmonella spp. tested showed that 38% were resistant to 4 or fewer antibiotics, and 62% were resistant to more than 4 antibiotics (see Tables 1 and 2).

Following exposure to YWCP, in general YWCP concentrations of 0.3-1.0% demonstrated a beneficial effect on E. coli antibiotic resistance patterns. Specifically, increased sensitivity of E. coli to ampicillin, chloramphenicol, streptomycin, and neomycin was seen as determined by the Kirby Bauer method, although no curing effect of YCWP was seen as determined by replica plating. In comparison, curing for amoxicillin and clavulanic acid (100%), chloramphenicol (100%), and ampicillin (5%) was seen when isolates were treated with ethidium bromide (EB) or iododeoxyuridine (IDU), whereas no curing for tetracycline and neomycin was seen for EB or IDU treatment.

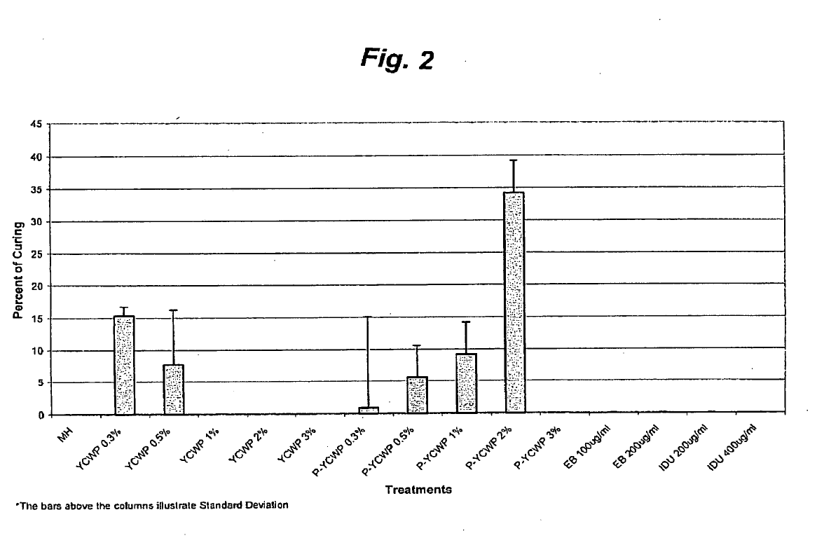

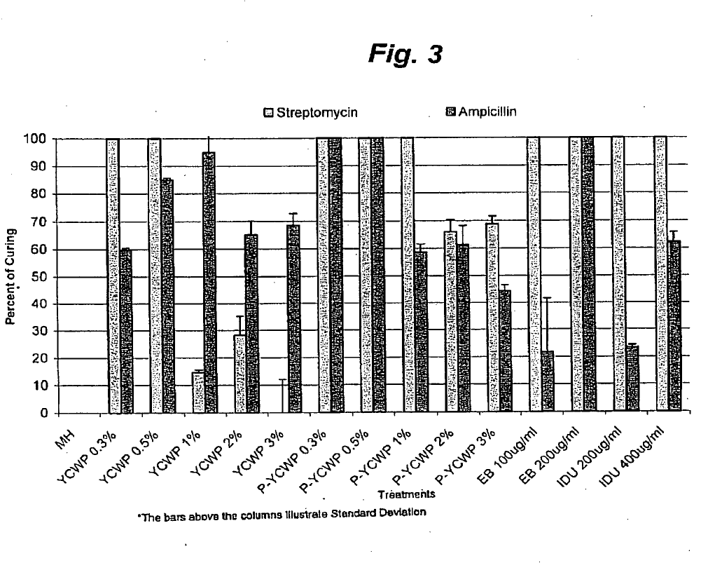

In contrast, S. enteritidis, S. Montevideo, S. schwarsengrund, and S. senf showed significant curing following exposure to P-YCWP or YWCP. Results were comparable to exposure to EB and IDU. Most of the P-YCWP concentrations evaluated showed some percentage of curing for S. enteritidis, with 2% P-YCWP being most effective (see FIG. 2). The YCWP evaluated at 0.3% and 0.5% showed some curing of S. enteritidis also. Neither EB nor IDU showed any curing capacity for S. enteritidis (FIG. 2). On the other hand, curing of S. Montevideo, S. schwarzengrund, and S. senf was seen only with EB (200 μg/ml; data not shown). Similarly, exposure of S. Montevideo to 0.3 and 0.5% P-YCWP or YCWP (FIG. 3) resulted in favorable curing results. Treatment with YCWP resulted in curing results comparable to EB and IDU.

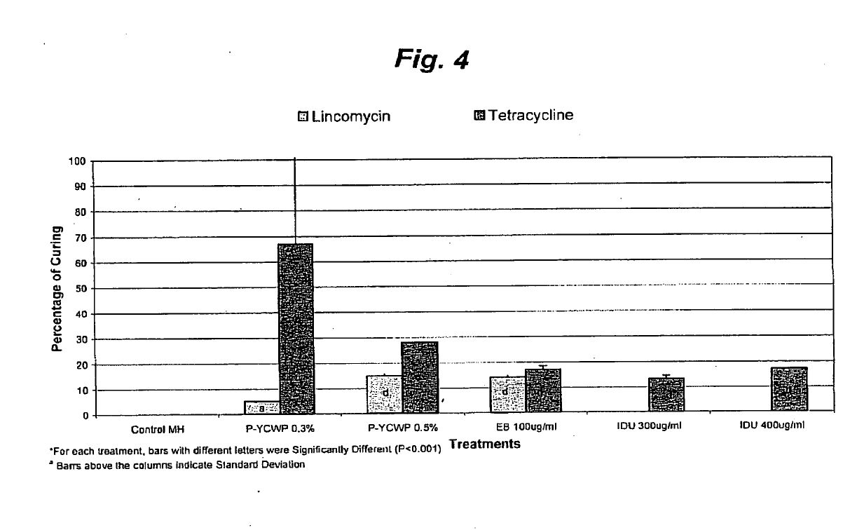

For comparison, commercial E. coli strains containing a narrow range plasmid (E. coli XL1-blue) and a broad range plasmid (E. coli ATCC 33766, plasmid pRK248clts) were included in the curing protocol. Both strains carried a tetracycline-resistant determinant (TeR), and both were resistant to clindamycin, penicillin, lincomycin, and vancomycin. Curing experiments were conducted as described above. Neither yeast cell wall preparations nor traditional curing agents (EB, IDU) cured the organism carrying the broad range plasmid (data not shown). On the other hand, for the organism containing the narrow range plasmid, curing was seen with P-YCWP at 0.3 and 0.5%, as well as EB and IDU (FIG. 4). Further, treatment of E. coli XL1-blue with 0.3% P-YCWP resulted in a 5% increase in sensitization to lincomycin and a 68% increase in sensitization for tetracycline (FIG. 4). Recovery of sensitivity to various antibiotics, and concomitant plasmid loss, was seen in other gram-negative isolates following exposure to yeast cell wall (Table 4). Indeed, curing of selected Salmonella spp. by yeast cell wall exposure was confirmed to be accompanied by loss of plasmids (ranging from a single plasmid to all 7) in comparison to untreated isolates (Table 4).

TABLE 4
Selected gram-negative isolates demonstrating plasmid loss or
curing following exposure to yeast cell wall preparations.

| | # Plasmid | Plasmid Size | Antibiotic Sensitivity |
|---|---|---|---|
| Strains | Lost | (Kb) | Recovered |
| E. coli XL1-Blue | 1 | 2.5 | Lincomycin and |
| | | | Tetracycline |
| S. enteritidis | 1 | 2.5 | Streptomycin |
| S. senft | 2 | 2.5, 7 | Streptomycin |
| S. schwarzengrud | 1 | 1 | Ampicillin |
| S. monterido | 6 | 1, 1.5, 2, | Ampicillin, |
| | | 2.5, 3.5, 7 | Streptomycin |

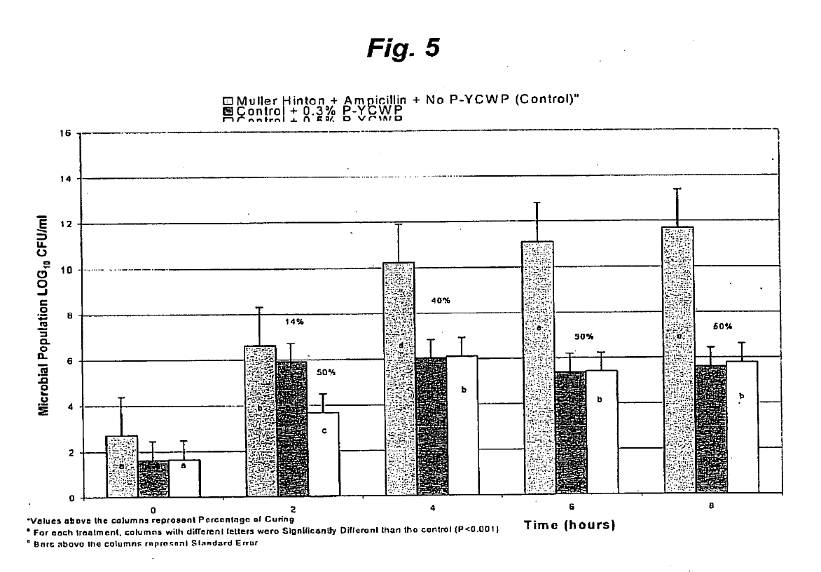

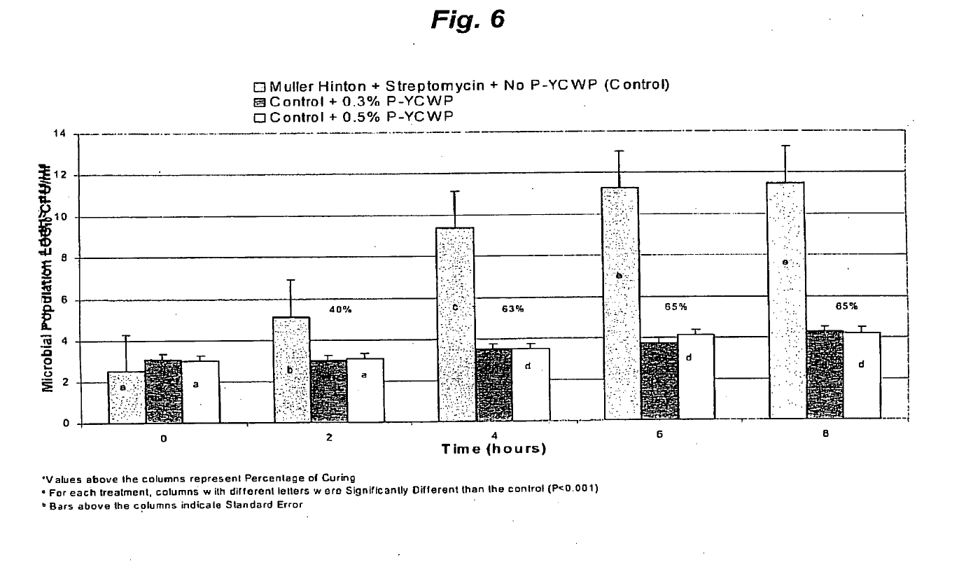

FIGS. 5 and 6 show percent curing over time for ampicillin (FIG. 5) and streptomycin (FIG. 6) resistant Salmonella spp., following exposure to P-YCWP (0.3%, 0.50%) and growth on media containing 32 μg/ml ampicillin or 1000 μg/ml streptomycin, respectively. It is noted that no effect on growth of isolates was observed on agar plates without ampicillin or streptomycin, as appropriate (data not shown). Exposure to P-YCWP resulted in a 4-log reduction in microbial population at 4 hours of incubation, after which the population reduction was more than 5-log. Streptomycin-resistant Salmonella isolates grown in media with streptomycin showed an approximately 8-log reduction over time. Thus, in comparison to control cultures, approximately 65% curing was seen for the streptomycin-resistant Salmonella isolates.

### Example 2

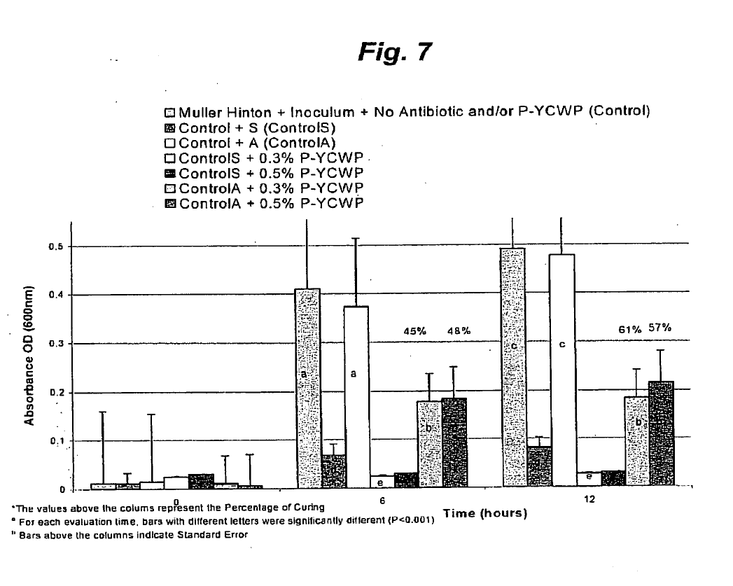

To determine whether the yeast cell wall preparations were deactivating antibiotic via an adsorption mechanism, two groups of Salmonella isolates were treated with P-YCWPs with or without selected antibiotics. The first isolate group was streptomycin-resistant and ampicillin-sensitive. With reference to FIG. 7, growth in the presence of streptomycin (1000 μg/ml) demonstrated a decrease in absorbance compared to antibiotic-free controls. Growth in the presence of ampicillin (32 μg/ml) did not affect growth rate compared to controls. Including 0.3 and 0.5% P-YCWP did not alter sensitivity patterns in the presence of streptomycin, but isolates did not grow. Had antibiotic been adsorbed by P-YCWP, the isolates would have grown as in the absence of antibiotic and P-YCWP.

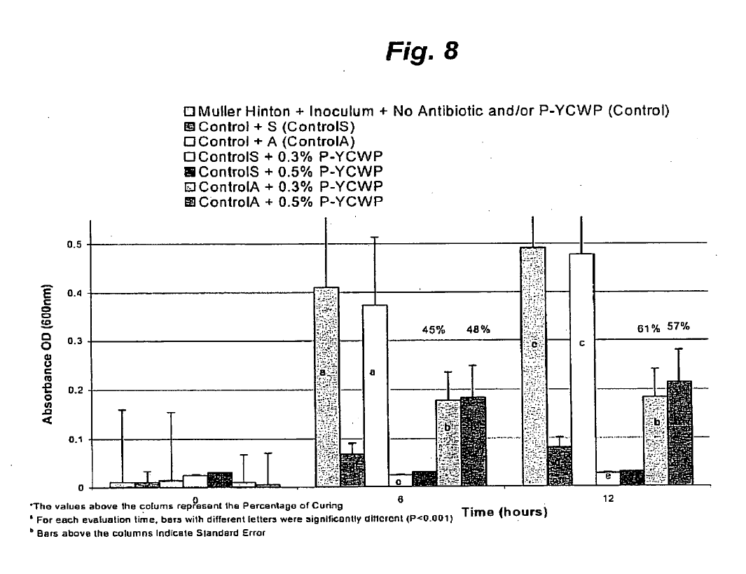

The second group of Salmonella isolates were ampicillin-resistant and streptomycin-sensitive. Results showed no growth in the presence of streptomycin, but growth in the presence of ampicillin (FIG. 8). Inclusion of P-YCWP and ampicillin resulted in a reduction in absorbance over time, meaning a progressive curing effect due to the P-YCWP exposure.

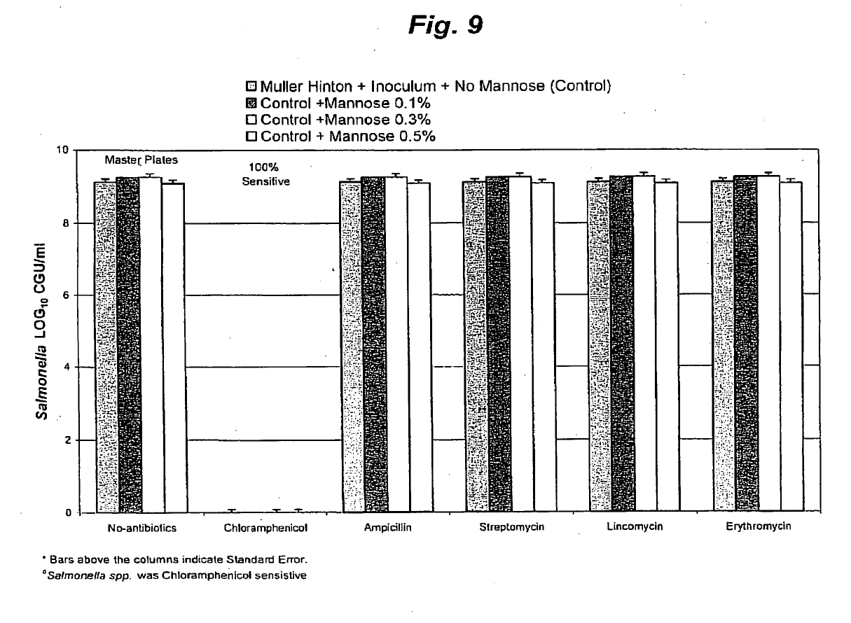

For comparison (FIG. 9), mannose was substituted in the curing protocols. The sensitivity pattern for Salmonella and commercial E. coli isolates (XL1-blue, ATCC 33766) was not affected by mannose treatment, thus there was no curing.

### Example 3

The majority of E. coli and Salmonella spp. evaluated agglutinated either P-YCWP or YCWP (Table 5).

TABLE 5
The effect of glucose, fructose and mannose on the ability of
E. coli and Salmonella spp. to agglutinate with YCWP or P-YCWP.

| | Glucose | Fructcose | Mannose | PBS + Bacteria + |
|---|---|---|---|---|
| Isolates | 100 mM | 100 mM | 100 mM | P-YCWP/YCWP |
| Salmonella spp. (13) | Positive (+) | Negative (−) | Negative (−) | Positive +/+ |
| S. Cholerasuis (2) | Negative (−) | Negative (−) | Negative (−) | Negative −/− |
| S. pullorum (1) | Negative (−) | Negative (−) | Negative (−) | Negative −/− |
| E. coli (30) | Positive (+) | Negative (−) | Negative (−) | Positive +/+ |

P-YCWP/YCWP (0.1%) + plain PBS is a negative control (no agglutination)
P-YCWP/YCWP + PBS with bacteria (If bacteria adhere to it, there will be agglutination)

Thus, the above results show that the yeast cell wall preparations of the present invention provide an alternative method for curing antibiotic resistant enterobacteria. No effect on bacterial growth was observed, possibly because the evaluated isolates lack the necessary enzymes to metabolize complex oligosaccharides. Most of the Salmonella isolates exposed to 0.3 and/or 0.5% of the yeast cell wall preparations recovered sensitivity to certain antibiotics to which they were previously resistant. Indeed, certain isolates (S. enteritidis) recovered sensitivity to streptomycin upon exposure to yeast cell wall preparations, when in comparison the traditional curing agents EB and IDU had no effect. The present results were not mediated by adsorption of antibiotic, and did not appear to be affected by inclusion of mannose in the curing protocols. Thus, the present mode of action does not appear to be mediated by the mannose content of yeast cell wall. Without wishing to be bound by any particular theory, it may be that the present effects result partially from disruption of antibiotic-resistance plasmid transfer (blocking) between bacteria, plus dilution of the antibiotic-resistant group by the non-antibiotic resistant group over time. The latter phenomenon may be related to a decrease in plasmid-containing bacteria (curing). Accordingly, YCWP may be a potential curing agent for antibiotic resistance in certain enteric pathogens, may be an important tool for overcoming antibiotic resistance in such organisms, and may provide a natural strategy to supplement traditional therapies for control of bacterial infection without induction of antibiotic resistance.

### Example 4

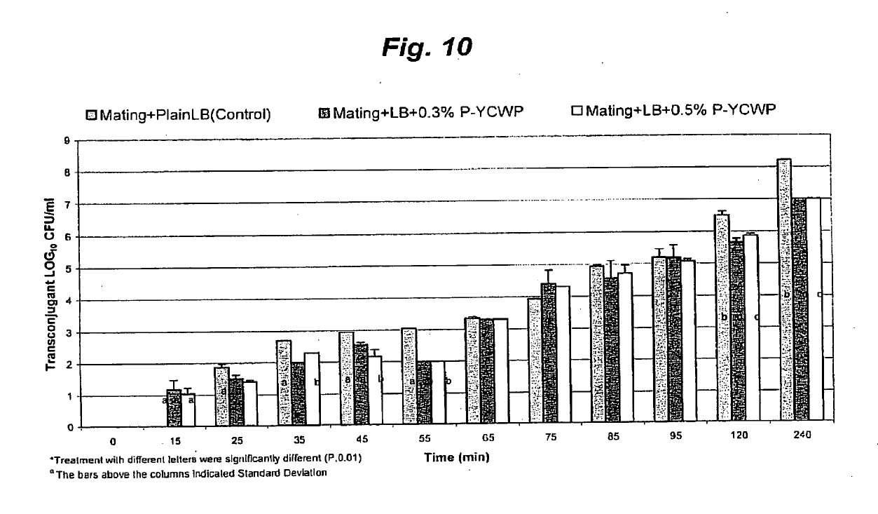

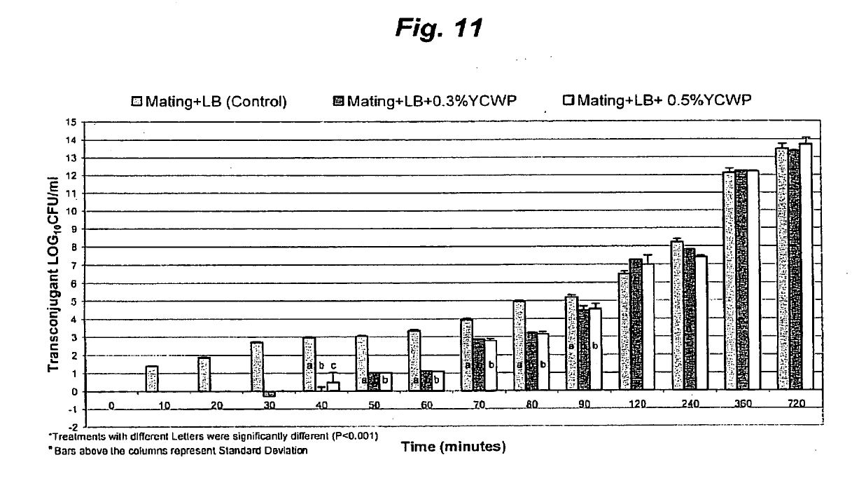

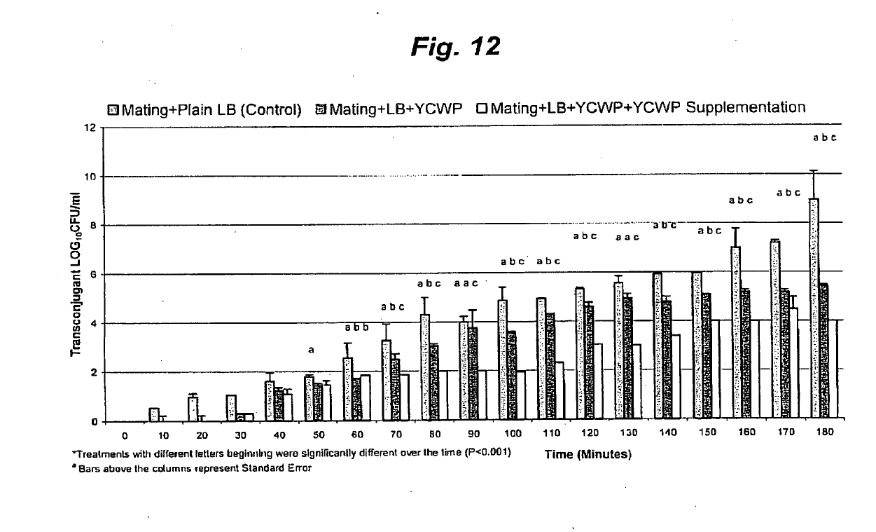

Further studies were undertaken to evaluate effect of yeast cell wall preparations on bacterial attachment and genetic transfer of antibiotic resistant plasmids. Mating experiments using E. coli XL1-Blue (donor) and E. coli MC1000 (recipient) isolates in the presence of P-YCWP showed that transconjugant formation (colony growth on MacConkey agar containing tetracycline and ampicillin) was reduced for the first 55 min following exposure. Both P-YCWP and YCWP preparations inhibited transconjugant formation during the initial growth phases (FIGS. 10 and 11). Supplementation of fresh YCWP (0.5%) to mating cells at 60 and 120 min. further delayed transconjugant formation, and kept the transconjugant population 1 to 2 logs lower than observed without YCWP supplementation (FIG. 12). Increasing the number of recipients did not significantly affect transconjugant formation (data not shown).

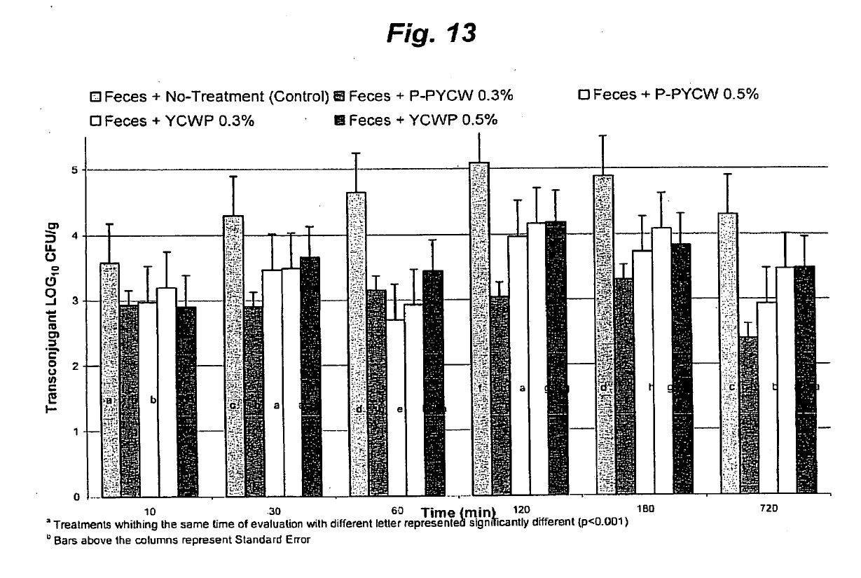

In solid media (LB agar) no growth of transconjugant was seen. On the other hand, isolate mating experiments in swine feces (simulating a natural growth environment) in the presence of P-YCWP or YCWP resulted in a significant reduction in transconjugant formation over time compared to controls (FIG. 13). P-YCWP (0.3%) was most efficient. Comparatively, P-YCWP was most efficient in reducing transconjugant formation in the swine feces model. Accordingly, an effective strategy for control and reduction of transconjugant formation between isolates is provided, thus providing an effective method for prevention of transfer of antibiotic-resistance without risking harm to desirable bacterial microflora.

### Example 5

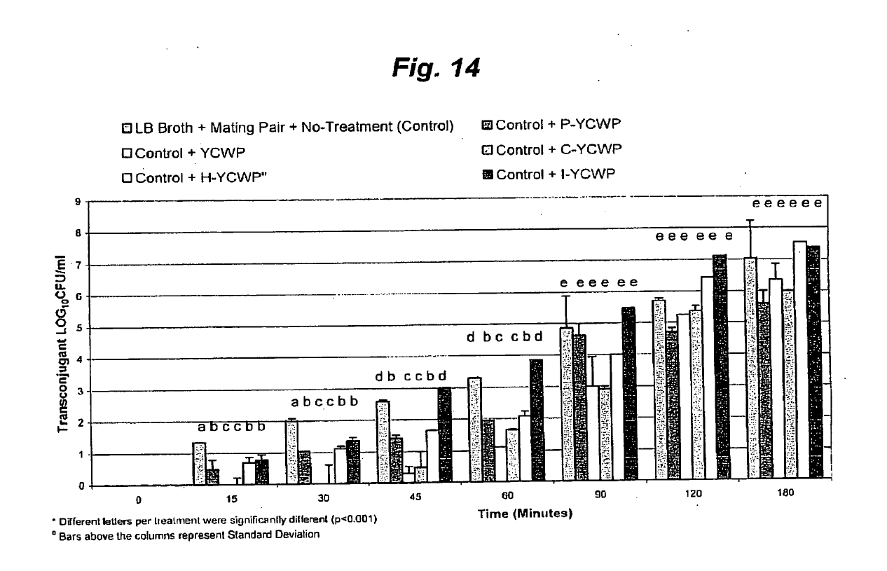

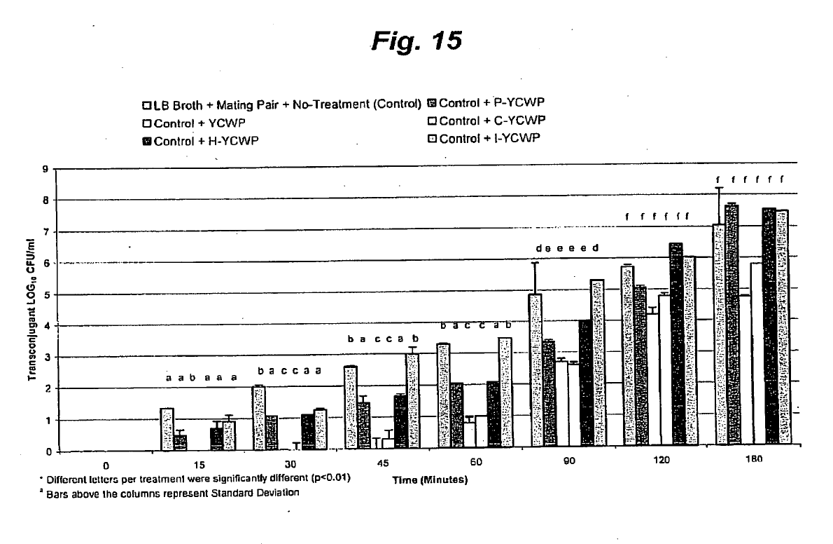

Multiple yeast cell wall formulations (see Table 3) were evaluated for ability to control, prevent, or minimize conjugation between donor and recipient bacterial isolates. As described above, commercial E. coli donor and recipient isolates were used as a model. Following in vitro mating, transconjugants were obtained from antibiotic selective media. None of the yeast cell wall preparations affected growth of donor or recipient isolates. Most of the yeast cell wall preparations evaluated showed a significant effect on transconjugant formation, some comparable to P-YCWP and YCWP (see FIGS. 14 and 15).

Accordingly, YCWP is shown to be a natural alternative for effectively inhibiting conjugation, thereby decreasing antibiotic resistance transfer among microorganisms. A strategy for controlling or treating multi-drug resistance in bacteria, and for controlling or preventing transfer of antibiotic resistance to or from food animals and humans, is therefore provided.

### Example 6

To evaluate whether oral provision of a composition containing yeast cell wall (BIO-MOS, Alltech, Inc.) was effective in reducing antibiotic resistance in bacteria, broiler chickens were randomly assigned to 16 pens of four birds each. Pens were randomly split into two groups of eight pens (32 birds/group). Treatments were as follows:

1. Control (no supplementation);
2. Supplemented (BIO-MOS; 1 kg/T of feed).

On sampling days, 2 birds from each pen were randomly selected, humanely euthanized, and cecal contents were recovered and lyophilized. DNA from cecal contents (0.05 g) was extracted and purified using a high pure PCR purification kit (Roche Diagnostics, GmBH, Penzburg, Germany) according to the manufacturer's instructions.

Primers (see Table 6) were designed to amplify a 200400 bp fragment of the tetracycline resistance genes tet A, tet B, tet L, and tet M (prevalent in Escherichia, Salmonella, Clostridium, and Enterococci). The genes were then cloned and sequenced using BLAST to confirm identity. After sequence confirmation, real-time PCR primers were designed using primer express. Cloned PCR products were purified using the genelutetm plasmid mini-prep kit (Sigma-Aldrich, Inc., St. Louis, Mo.). DNA concentration was quantified by UV spectrophotometry. The copy number of each DNA standard was calculated based on mass concentration and the average molecular weight of each tet gene.

TABLE 6
Real-time PCR primers for tet genes.

| | Forward Primer | Reverse Primer |
|---|---|---|
| Gene | 5′-3′ | 5′-3′ |
| tet A | ctt gct aca tcc | gta gtt gag agc ctg |
| | tgc ttg cct | tcc cgt |
| tet B | cag tgc tgt tgt | gct tgg aat act gag |
| | tgt cat taa | tgt aa |
| tet L | ctg cat ttc cag | cct ttg gaa tat agc |
| | cac tcg taa t | cgc ca |
| tet M | tcg ttt ccc tct att | gta atc aaa cag aag |
| | acc gta tcc | gta gaa ctg tat cct |

To evaluate effect of supplementation with yeast cell wall preparation, real-time PCR of cecal samples (16 from control group, 16 from supplemented group) was performed. Triplicate real-time PCRs were run using the ABI 7500 real time PCR system. Reaction conditions were one cycle 50° C. for 2 mins, one cycle 95° C. for 10 mins, and 42 step-cycles of 95° C. for 15 sec., 60° C. for 1 min. Gene copy numbers were log-transformed for statistical analysis.

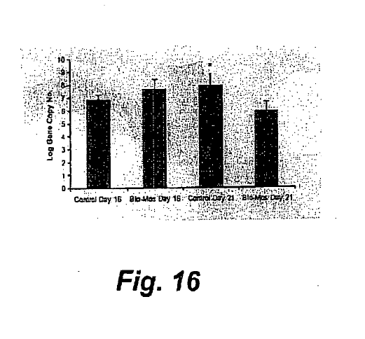

A significant reduction in tet A gene number was seen in the supplemented group by day 21 of supplementation (FIG. 16). No significant effect was seen on tet B, tet L, or tet M gene numbers during the 21 d sampling period (data not shown).

### Example 7

The experiment set forth in Example 6 was repeated and extended using turkeys, to evaluate the effect of more extended periods of supplementation of a yeast cell wall containing composition (BIO-MOS, Alltech, Inc.). Primers and real-time PCR assays were as set forth in Example 6. Turkeys were randomly assigned to 16 pens (6 birds/pen) and supplemented from day 1 to day 42 of age (BIO-MOS, 1 kg/T). On each of three sampling days (day 28, day 35, day 42), two birds per pen were randomly selected, humanely euthanized, and cecal contents recovered and processed as set forth in Example 6.

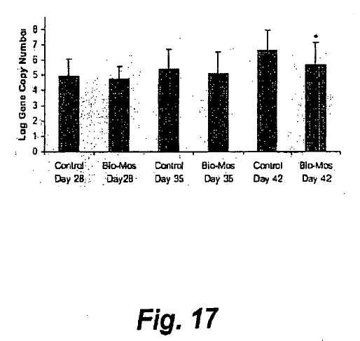

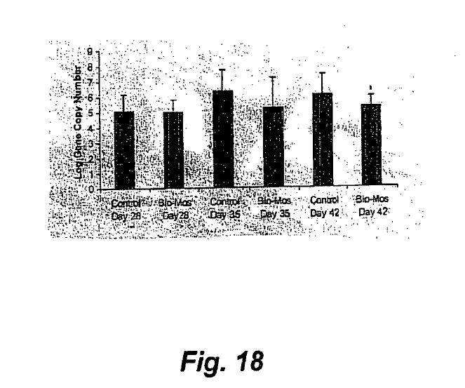

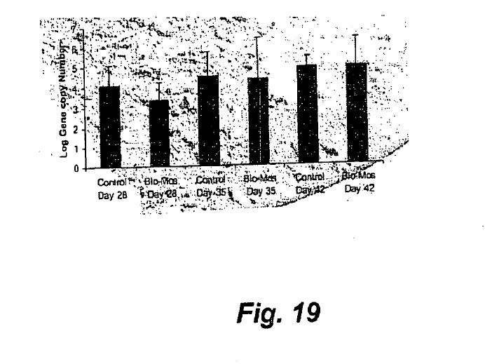

Significant reductions in tet A and tet B gene numbers were seen at day 42 of treatment (FIGS. 17 and 18). A significant decrease in tet M gene numbers was seen at day 28, but had disappeared by day 35 (FIG. 19). No effect on tet L gene numbers was seen (data not shown). Thus, supplementation of feed with a yeast cell wall containing composition reduced the load of a sub-group of tetracycline-resistant bacteria in the cecal contents of turkeys. The effect was most pronounced after 42 days of supplementation.

It is accordingly shown that the present invention provides a method and a composition for restoring antibiotic sensitivity of bacteria, and for preventing or reducing transfer of antibiotic resistance between bacteria. Still further, the present invention provides a method for reducing numbers of antibiotic resistance genes in animals, comprising providing a yeast cell wall-containing composition as a feed or feed supplement.

Additional advantages, and other novel features of the invention will become apparent to those skilled in the art upon examination of the foregoing disclosure, or may be learned with practice of the invention. The foregoing description of the preferred embodiment of the invention has been presented for purposes of illustration and description. It is not intended to be exhaustive or to limit the invention to the precise form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments with various modifications as are suited to the particular use contemplated. All such modifications and variations are within the scope of the invention as determined by the disclosure and the appended claims, when interpreted in accordance with the breadth to which they are fairly, legally, and equitably entitled.

### CITATIONS OF LITERATURE

Andrup, L. and K. Anderson, 1999. A comparison of kinetics of plasmid transfer in the conjugation systems encoded by the F plasmid from Escherichia coli and plasmid pCF10 from Enterococcus faecalis. Microbiology 145: 2001-2009.
Andrup, L., L. Smidt, L. Anderson, and L. Boe. 1998. Kinetics of conjugative transfer: a study of the plasmid pXO16 from Bacillus thuringiensis subsp. Israelensis. Plasmid 40: 30-43.
Chakrabartty, P. K., A. K. Mishra, and S. K. Charabarti. 1984. Loss of plasmid linked drug resistance after treatment with iodo deoxy uridine. Indian J. of Exp. Biol. 22: 333-334.
Gupta, T. D., T. Bandyopathyay, S. G. Dastidar, M. Bandopadhyay, A. Mitra, and A. N. Charabarty. 1980. R plasmids of Staphylococcus and their elimination by different agents. Indian J. Exp. Biol. 18: 478-481.
Hahn, F. E., and J. Ciak. 1976. Elimination of resistance determinants from R-factor R1 by intercalating compounds. Antimicrob. Agents Chemotherapy 9: 77-80.
Kruse, H., and H. Sorum. 1994. Transfer of multiple drug resistance plasmids between bacteria of diverse origins in natural environments. Appl. Env. Microbiol. 60(11): 4015-4021.
Lakshmi, V. V., S. Padma, and H. Polasa. 1987. Elimination of multidrug-resistant plasmid in bacteria by plumbagin, a compound derived from a plant. Curr. Microbiol. 16: 159-161.
Lakshmi, V. V., and Thomas C. M. 1996. Curing of F-like plasmid TP181 by plumbagin is due to the interference with both replication and maintenance functions. Microbiology-UK 142: 2399-2406.
Morehead, M. C. and K. A. Dawson. 1992. Some growth and metabolic characteristics of monensin-sensitive and monensin-resistant strains of Prevotella (Bacteroides) ruminicola. Appl. Env. Microbiol. 58: 1617-1623.
National Committee for Clinical Laboratory Standards (NCCLS) 2000a. Methods for dilution of antimicrobial testing susceptibility test for bacteria that grow aerobically. Approved Standard M7-A5. National Committee for Clinical Laboratory Standards, Villanova, Pa.
National Committee for Clinical Laboratory Standards (NCCLS) 2000b. Performance standards for antimicrobial disk susceptibility tests. Approved Standard M7-A7. National Committee for Clinical Laboratory Standards, Villanova, Pa.
Obaseiki-Ebor, E. E. 1984. Rifampicin curing of plasmids in Escherichia coli K-12 rifampicin resistant host. J. Pharm. Pharmacol. 36: 467-470.
Poppe, C. and C. L. Gyles. 1988. Tagging and elimination of plasmids in Salmonella of avian origin. Vet. Microbiol. 18: 73-87.
Reddy, G., Shridbar, P., and H. Polasa. 1986. Elimination of Col E1 (pBR322 and pBR329) plasmids in Escherichia coli on treatment with hexamine ruthenium (III) chloride. Curr. Microbiol. 13: 243-246.
Trevor, J. T. 1986. Plasmid curing in bacteria. FEMS Microbiology Reviews 32(34): 149-157.

What is claimed is:

1. A method for reducing or eliminating antibiotic resistance in bacterial populations, comprising exposing the bacteria to a composition comprising a yeast cell wall preparation in an amount effective for reducing or eliminating resistance of said bacteria to at least one antibiotic.

2. The method of claim 1, wherein the yeast cell wall preparation is included in the composition in an amount effective for reducing or eliminating the presence of a bacterial plasmid which confers resistance to said antibiotic.

3. The method of claim 1, wherein the yeast cell wall preparation is included in the composition in an amount effective for preventing or reducing the transfer between bacteria of a plasmid which confers resistance to said antibiotic.

4. The method of claim 1, wherein the yeast cell wall preparation is included in the composition in an amount of from about 0.01% (w/v) to about 1.0% (w/v).

5. The method of claim 1, wherein the yeast cell wall preparation is derived from a species selected from the group consisting of Saccharomyces, Candida, Kluyveromyces, Torulaspora, and mixtures thereof.

6. The method of claim 1, wherein the antibiotic is selected from the group of antibiotics consisting of ampicillin, bacitracin, clindamycin, gentamycin, erythromycin, kanamycin, penicillin, streptomycin, tetracycline, trimethoprim, chloramphenicol, sulfamethazole, vancomycin, and mixtures thereof.

7. The method of claim 1, wherein the bacteria is selected from the group of bacteria consisting of a normal enteric bacteria, an enteric pathogen, a disease-causing organism, and any combination thereof.

8. The method of claim 7, wherein the bacteria is selected from the group of bacterial species consisting of Escherichia, Salmonella, Clostridium, Enterococci, and combinations thereof.

9. A method for reducing prevalence of an antibiotic-resistant bacteria in an animal, comprising administering to said animal a composition comprising a yeast cell wall preparation in an amount effective for reducing or eliminating the presence of an antibiotic-resistant bacterial population in said animal.

10. The method of claim 9, wherein the yeast cell wall preparation is derived from a species selected from the group consisting of Saccharomyces, Candida, Kluyveromyces, Torulaspora, and mixtures thereof.

11. The method of claim 9, wherein the composition is administered to said animal as a dietary supplement.

12. The method of claim 9, wherein the composition is formulated for admixing with a feed ration for said animal.

13. The method of claim 9, wherein the composition comprising said yeast cell wall preparation is administered to the animal in an amount providing yeast cell wall preparation at from about 0.5 to about 1.5 kg/T of feed.

14. The method of claim 9, wherein the composition is formulated for feeding to bovine, porcine, avian, equine, ovine, lapine, and caprine species.

15. The method of claim 14, wherein the avian species is a chicken, turkey, duck, goose, pheasant, quail, or a companion bird.

16. The method of claim 9, wherein the antibiotic is selected from the group of antibiotics consisting of ampicillin, bacitracin, clindamycin, gentamycin, erythromycin, kanamycin, penicillin, streptomycin, tetracycline, trimethoprim, chloramphenicol, sulfamethazole, vancomycin, and mixtures thereof.

17. The method of claim 9, wherein the bacteria selected from the group of bacteria consisting of a normal enteric bacteria, an enteric pathogen, a disease-causing organism, and any combination thereof.

18. The method of claim 17, wherein the bacteria is selected from the group of bacterial species consisting of Escherichia, Salmonella, Clostridium, Enterococci, and combinations thereof.

19. The method of claim 9, wherein the yeast cell wall preparation is administered in conjunction with a conventional antibiotic therapy.


SEQUENCE LISTING (8 sequences)

SEQ ID NO: 1 - DNA, 21 bp, Artificial
  real time PCR primer
cttgctacatcctgcttgcct

SEQ ID NO: 2 - DNA, 21 bp, Artificial
  real time PCR primer
gtagttgagagcctgtcccgt

SEQ ID NO: 3 - DNA, 21 bp, Artificial
  real time PCR primer
cagtgctgttgttgtcattaa

SEQ ID NO: 4 - DNA, 20 bp, Artificial
  real time PCR primer
gcttggaatactgagtgtaa

SEQ ID NO: 5 - DNA, 22 bp, Artificial
  real time PCR primer
ctgcatttccagcactcgtaat

SEQ ID NO: 6 - DNA, 20 bp, Artificial
  real time PCR primer
cctttggaatatagccgcca

SEQ ID NO: 7 - DNA, 24 bp, Artificial
  real time PCR primer
tcgtttccctctattaccgtatcc

SEQ ID NO: 8 - DNA, 30 bp, Artificial
  real time PCR primer
gtaatcaaacagaaggtagaactgtatcct